(19) United States
(12) Patent Application Publication
(10) Pub. No.: US 2023/0190781 A1
(43) Pub. Date: Jun. 22, 2023

(54) ADEMETIONINE AND CANNABIDIOL BASED SOLID PHARMACEUTICAL COMPOSITION AND METHOD FOR PREPARING IT

(71) Applicant: Mario Antonio BASILE, Baranzate (IT)

(72) Inventor: Mario Antonio BASILE, Baranzate (IT)

(21) Appl. No.: 17/925,761

(22) PCT Filed: Dec. 15, 2021
(86) PCT No.: PCT/IB2021/061748

§ 371 Date: Nov. 16, 2022

(30) Foreign Application Priority Data
Dec. 15, 2020 (IT) 102020000030914

(51) Int. Cl. A61K 31/7076; A61K 9/00; A61K 31/05; A61K 31/352; A61K 9/48; A61K 9/20; A61K 47/10; A61K 9/28

(52) U.S. Cl. CPC A61K 31/7076; A61K 9/0053; A61K 31/05; A61K 31/352; A61K 9/485; A61K 9/4858; A61K 9/2013; A61K 9/2009; A61K 9/2054; A61K 47/10; A61K 9/2846

(57) ABSTRACT

A solid pharmaceutical composition, with no narcotic action, for the oral administration of ademetionine and cannabidiol, to the method for preparing it, and to the pharmaceutical, nutraceutical or veterinary formulations containing it.

### TECHNICAL FIELD

The invention relates to a solid pharmaceutical composition for oral use, with no narcotic effect, to the method for preparing it and to the pharmaceutical, nutraceutical and veterinary formulations containing it.

### BACKGROUND

Ademetionine (S,S-adenosyl-L-methionine), marketed as Samyr®, is a drug for disorders of the central nervous system and in particular for depressive states; it is also used in the treatment of inflammatory diseases, for example in osteoarthritis. Ademetionine is also available in nutritional supplements for both humans and animals.

The S-adenosyl-L-methionine molecule is an ion characterised by strong chemical instability, only its salts with strong acids are stable. The mixed sulphate/p-toluene sulphate salt, in which 50-55% is ademetionine ion, while 45-50% is made up of the two acids, is commercially available. It is highly hygroscopic and deliquescent, which is a problem in industrial processings: they must be carried out in a strongly dehumidified environment, typically with relative humidity (RH) of less than 20%. There are also preparations that mix the active substance with excipients, e.g. dehydrators, to improve stability, as reported in EP2170920 and US2011/0300081.

Cannabidiol (CBD) is a plant product, contained in Cannabis sativa, which is known both as an industrial crop and for recreational use. CBD has no narcotic properties and is generally extracted from non-narcotic hemp varieties (industrial hemp), or obtained by chemical synthesis. The psychotropic effects of Indian hemp (marijuana) are instead attributable to delta-9-tetrahydrocannabinol (THC). Other substances with a terpenic structure, known as cannabinoids (or phytocannabinoids), are also normally present in the extracts obtained from hemp. The consumption of Cannabis preparations is generally prohibited, but in some states, CBD preparations are considered legal as long as the THC content is below a certain limit, e.g. below 1%.

In practice, commercial CBD preparations can be divided into two groups:
1) purified hemp extracts, usually in the form of oil, fluid extract, or dry extract. They may contain other phytocannabinoids but have a low THC content;
2) pure cannabidiol, in the form of an amorphous powder or crystalline solid. In pure CBD, the presence of THC and other cannabinoids is absolutely negligible.

CBD is currently taken for the treatment of various pathologies, in particular as an adjuvant in anticancer therapies, for the improvement of appetite and to counteract the psychotropic effects of THC. Pure cannabidiol is marketed in the USA as Epidiolex®, while in Italy CBD is present in the drug Sativex®, an ethanolic solution of CBD and THC in almost equal parts. The composition of Sativex is obviously unrelated to the purposes of the patent, given the significant presence of THC. In other preparations, attempts are made to promote the enteric absorption of CBD, which is not very soluble in water, for example by means of the formulation with oleosomes, as described in patent WO2020/124268, or with mixtures of phospholipids and polyethylene glycols, as described in patent AT509000.

The association of ademetionine and cannabidiol is described in forms of administration by transdermal absorption, for example in patches as described for example in US2019/0216745. However, in this composition it is not excluded that THC is present initially or is formed later.

An analysis of the uses of both ademetionine and CBD can be found, for example, in Levine et al, Brain Behaviour and Immunity, vol. 85, pages 152-161, March 2020, which evaluates the two active substances (in addition to a third one) separately and not in associated administration.

The association of ademetionine and CBD in the same pharmaceutical form presents considerable technical problems, given the strong acidity and hygroscopicity of ademetionine, which is very soluble in water, and the presence of CBD, which is instead soluble in oil and has a very low enteric absorption. In addition, CBD has photostability issues, as reported in AT509000.

A further problem is the high acidity of the ademetionine salts: it is known, in fact, that cannabidiol is not stable and degrades in the presence of acids (Trofin et al, Rev Chem (Bucharest) 63, 4, 422-427, 2012). In addition, the degradation of CBD in an acidic environment leads to the formation of THC (Merrick et al, Cannabis Cannabinoid Research 1.1, 102-112, 2016). This is a problem for the preservation of CBD formulations: over time, in addition to the decrease in the active substance (CBD), they can develop the presence of THC at levels higher than what is permitted by law.

It is therefore a question of combining two active substances with opposite characteristics: ademetionine is stable only in a strongly acidic environment, CBD is unstable in an acidic environment. It is therefore difficult to avoid degradation of the active substances.

In addition, if a formulation for oral use without narcotic activity is desired, THC formation must be avoided throughout the shelf-life of the formulation, which can be up to three years.

The new invention solves the problems of stability and incompatibility of the two active substances.

### SUMMARY

It is an object of the present invention to solve the above-mentioned problems of the prior art.

This and other purposes are achieved by means of a composition incorporating the characteristics which are the subject-matter of claim 1.

According to a further aspect, the invention is directed to a pharmaceutical formulation containing the aforesaid composition.

According to a further aspect, the invention is also directed to a method for preparing a pharmaceutical composition incorporating the characteristics of claim 10.

### DETAILED DESCRIPTION OF THE EMBODIMENTS

The combination in therapy of ademetionine and CBD in a form of oral administration without narcotic effect could be of great benefit in the treatment of various pathologies, particularly those related to the nervous system, e.g. in cases of depression, anxiety, sleep disorders. Furthermore, in osteoarticular inflammation it could be useful to associate the long-term therapeutic efficacy of ademetionine with the immediate benefit of CBD in the treatment of the associated pain. Unfortunately, such a formulation has not been developed so far, due to the technical problems described above.

A solid, stable composition with no narcotic activity, consisting of an ademetionine salt and a solution of cannabidiol in a fat or wax, has now been obtained: this composition overcomes the technical difficulties of preparing the usual pharmaceutical forms such as tablets, capsules and orosoluble granules, while improving the stability of both active substances. It therefore allows the simultaneous administration of ademetionine and cannabidiol in a single pharmaceutical formulation. Furthermore, it allows a good flexibility in dosages, as the two active substances can be formulated in widely variable proportions. Furthermore, it has the additional advantage of being not very hygroscopic.

In its preferred embodiment, the invention consists of a composition having, as a whole:
ademetionine content, expressed as ademetionine-ion, ranging between 10% and 55% of the total weight;
cannabidiol content between 1% and 40% of the total weight;
wax or fat content less than 30% of the total weight.

For the purposes of the invention, both purified hemp extracts and pure cannabidiol, obtained by extraction or chemical synthesis, may be used. A high level of purity of cannabidiol preparations is not required, it is sufficient to adjust the quantity of the preparation based on the content of the active substance (cannabidiol), as long as the THC content is lower than the legal limits.

Advantageously, the composition may also comprise other components such as phospholipids, surfactants, lubricants, sweeteners, flavourings and other pharmaceutically accepted excipients.

In a preferred embodiment, the composition is presented in solid form as a granulate; in a particularly preferred form, said granulate is characterised by a humidity content of less than 5.0%. In a possible embodiment, said granulate is dosed in stick-packs or in capsules. In a further embodiment of the invention, said granulate is used for the preparation of plain or film-coated tablets.

In further embodiments, said granulate may contain other active substances: for example unsaturated omega-3/6/9 fatty acids, fat-soluble vitamins such as vitamin A, vitamin E, ascorbyl palmitate, carotenes, or other plant extracts, in particular: chamomile, hawthorn, valerian, St. John's wort, tea.

In a possible embodiment, the invention consists in the process for obtaining a stable composition of ademetionine and fatty matter. In a further aspect, the invention consists in the process for drying said composition.

The method of the invention comprises:
a) dosing the fatty matter;
b) dosing cannabidiol,
c) dosing any other active substances and/or excipients;
d) dissolving the compounds dosed under points b) and c) in the fatty matter, possibly heating up to melting;
e) dosing ademetionine;
f) mixing ademetionine with the mixture obtained under point d);
g) keep stirring for a time sufficient to homogenise the mixture;
h) optionally, cooling the composition below the melting temperature of the fatty matter;
i) optionally, screening the obtained compound to ensure homogeneity of particle size.

The methods described above can be carried out in any order of succession.

Optionally, the processing is carried out at a temperature equal to or higher than +64° C., the melting point of cannabidiol.

Preferably, for a better drying of the granulate, step g) is carried out under vacuum, at a temperature equal to or higher than 20° C.

The following examples illustrate the invention in greater detail.

### DEFINITIONS

A fatty matter is defined as a fat, wax or a mixture of fats approved for food or pharmaceutical use such as, for example: vegetable oil, animal or vegetable butter, margarines or other hydrogenated fats, purified triglycerides, mono- or di-glycerides of fatty acids, polyethers such as polyethylene or polypropylene glycols, polyalcohols and the esters thereof with fatty acids. Preferably, said fatty matter has a melting point ranging between +30° C. and +80° C., more preferably between +35° C. and +70° C.

A pharmaceutically acceptable excipient is defined as an excipient useful in the preparation of a pharmaceutical formulation and generally regarded as safe, non-toxic and administrable to humans and animals.

### EXAMPLE 1

Preliminary tests to study the formulations: an ademetionine salt was mixed with various fatty matters and the stability of the active substance was evaluated.

For each experiment, 485 g of mixed sulphate/p-toluenesulphonate salt with 51.5% (w/w) of ademetionine-ion, corresponding to 250 grams of active substance, mixed with the components shown in Table 1, were used.

TABLE 1
Compositions of ademetionine and fatty matter

| Test | Composition | Amount |
|---|---|---|
| A | Stearin | 20 g |
| | Phosphatidylcholine | 10 g |
| B | Stearin | 30 g |
| C | Stearin | 20 g |
| | Soya lecithin | 10 g |
| D | Stearic acid | 30 g |
| E | Olive oil | 20 g |
| | Soya lecithin | 10 g |
| F | MCT | 30 g |
| | Soya lecithin | 10 g |
| | Sucrose esters | 10 g |
| G | Phosphatidylcholine | 40 g |
| H | Soya lecithin | 50 g |

Stearin is a mixture of purified oleic and stearic acids, MCT (medium chain triglycerides) is a mixture of purified medium chain triglycerides.

The general processing method comprises:
a) dosing the fatty matter and heat it until it melts;
b) dosing the excipients;
c) dissolving the excipients in the melted fatty matter;
d) dosing the ademetionine salt;
e) mixing the solution obtained under point c) with ademetionine;
f) keep stirring for about 30 minutes;
g) cooling the composition below +30° C.

In the case of test F it was sufficient to heat to +45° C., in the other tests to +60/70° C.; in test E no heating was necessary. All compositions were then screened on a 500-micron steel mesh sieve.

All preparations have proved to be low hygroscopic, free flowing and easy to process; the stability of ademetionine, as reported below, is more than satisfactory.

### EXAMPLE 2

The composition was prepared comprising:

| | |
|---|---|
| ademetionine | 250 g |
| CBD | 20 g |
| stearin | 50 g |
| soya lecithin | 10 g |

For ademetionine, the salt from Example 1 (480 g) was used, and for cannabidiol, a pure crystalline product with CBD 98% w/w (20.4 g) was used. The method of Example 1 was carried out, by dosing the CBD and the soya lecithin in the melted stearin.

A white powder was obtained containing ademetionine (as an ion) 44.1% by weight and CBD 3.6% by weight; THC was absent.

### EXAMPLE 3

By operating as described in Example 2, the composition was prepared comprising:

| | |
|---|---|
| ademetionine | 250 g |
| CBD | 40 g |
| stearic acid | 20 g |
| phosphatidylserine | 30 g |
| soya lecithin | 10 g |

Ademetionine was added to the melted mixture of stearic acid, soya lecithin and phosphatidylserine.

A white powder was obtained with ademetionine (ion) 42.8% w/w, CBD 6.7% w/w, THC absent.

### EXAMPLE 4

By operating as described in Example 2, the composition was prepared comprising:

| | |
|---|---|
| ademetionine | 250 g |
| CBD | 10 g |
| stearic acid | 20 g |
| phosphatidylserine | 30 g |
| sucrose esters | 10 g |

13 g of hemp oil extract refined by distillation was used, with CBD 77% and THC less than 0.2%, by weight.

A white powder was obtained with ademetionine 44.7% w/w and CBD 1.8% w/w, THC<0.05% w/w.

### EXAMPLE 5

By operating as described in Example 2, the composition was prepared comprising:

| | | |
|---|---|---|
| ademetionine | 250 | g |
| CBD | 25 | g |
| vegetable oil | 49.5 | g |
| phosphatidylserine | 30 | g |
| sucrose esters | 10 | g |

A 46% (w/w) ademetionine preformulation stabilised with calcium oxide and calcium chloride (543 g) was used; the distillate described in Example 4 (32.5 g) was used for CBD. The vegetable oil used is a mixture of extra virgin olive oil-, rich in polyphenols, and of linseed oil, rich in polyunsaturated fatty acids (omega-3/6/9).

A whitish powder was obtained with ademetionine ion 37.5%, CBD 3.7%, alpha-linoleic acid 3.7%, traces of THC less than 0.1% (all percentages by weight).

### EXAMPLE 6

By operating as described in Example 2, the composition was prepared comprising:

| | | |
|---|---|---|
| ademetionine | 152 | g |
| CBD | 150 | g |
| stearin | 50 | g |
| medium chain triglycerides | 134 | g |
| vegetable oil | 30 | g |
| phosphatidylcholine | 30 | g |

330 g of stabilised ademetionine and 200 g of distilled CBD already described in Example 4 were used.

A whitish powder was obtained with ademetionine ion 20% and CBD 20%, THC about 0.05% (% by weight).

### EXAMPLE 7

### Stability of Ademetionine in the Compositions

The compositions prepared according to the previous examples were placed in dark glass jars in cabinets with temperature and relative humidity control (+40° C. and 75% RH), periodically checking the content of active substances. The stability of the raw material is used as a reference.

TABLE 2
stability of ademetionine in the compositions

| | Time (months) | | | | % residual |
|---|---|---|---|---|---|
| Sample | 0 | 1 | 2 | 3 | ion |
| composition 1C | 48.5 | 48.2 | 47.8 | 47.4 | 97.9% |
| composition 2 | 44.1 | 43.8 | 43.5 | 43.1 | 97.7% |
| composition 4 | 44.7 | 44.3 | 44.1 | 43.9 | 98.2% |
| composition 5 | 20.6 | 20.5 | 20.3 | 20.1 | 97.6% |
| Ademetionine | 51.5 | 51.0 | 49.6 | 48.8 | 94.8% |
| Stabilised adm | 46.0 | 45.8 | 45.7 | 45.0 | 97.8% |

Stabilised adm: ademetionine stabilised with inorganic desiccants (see experiment 5).
% residual ion: active substance content after three months as a percentage with respect to time zero.

The results show that the preparation of Example 1 letter C and the compositions of experiments 2, 4 and 5 are more stable than ademetionine sulphate/p-toluene sulphate. The stability of compositions 2 and 4 is similar to that of the ademetionine preformulation with inorganic desiccants used in experiment 5. Furthermore, all the granulates obtained are low hygroscopic and have good flowability.

Mixing with a fatty matter gave stable and easily usable compositions of ademetionine and fatty matter, with or without cannabidiol.

### EXAMPLE 8

### Stability of Cannabidiol in the Compositions

The compositions described in Example 7 were also analysed for CBD and THC content. A mixture of equal parts of ademetionine sulphate/p-toluene sulphate and pure cannabidiol, mixed in the absence of fatty matter, was used as a reference.

TABLE 3
CBD and THC content in the compositions

| | Time (months) | | | | % residual |
|---|---|---|---|---|---|
| | 0 | 0 | 3 | 3 | CBD |
| Sample | CBD | THC | CBD | THC | — |
| composition 2 | 3.6 | >0.05 | 3.4 | 0.06 | 94.4% |
| composition 5 | 21.0 | 0.05 | 20.6 | 0.09 | 98.1% |
| Reference | 48.9 | 0.11 | 38.2 | 6.2 | 78.1% |

CBD and THC: content in mg/dose. residual CBD: active substance content after three months as a percentage with respect to time zero.

The results show how the cannabidiol in the compositions described above is stable, while it degrades in the reference mixture. Furthermore, the THC content in the compositions is always less than 0.5% by weight, whereas it increases significantly in the reference mix.

### EXAMPLE 9

### Preparation of the Capsules

The composition of Example 2 was used (with the appropriate excipients) for the preparation of capsules, dosing about 567 mg per capsule to obtain an average dosage of:

| | |
|---|---|
| ademetionine | 250 mg |
| CBD | 20 mg |
| colloidal anhydrous silica | 3 mg |
| magnesium stearate | 10 mg |

The capsules (double-zero measurement) were filled with a filling machine in a dehumidified environment at relative humidity of 40-45%, without encountering any difficulties or inconveniences.

### EXAMPLE 10

### Preparation of the Tablets

The compositions of Examples 2, 4 and 5 were used for the preparation of tablets with a punch machine, working in an environment at 40-45% RH.

Per single tablet, the following were dosed:

| | | |
|---|---|---|
| ademetionine | 250 | mg |
| CBD | 10-40 | mg |
| microcrystalline cellulose | 70 | mg |
| colloidal anhydrous silica | 3 | mg |
| magnesium stearate | 10 | mg |

Plain tablets of suitable hardness were obtained according to the European Pharmacopoeia standards.

The tablets prepared as above were subjected to filming in the drum mixer using a methacrylic acid copolymer equal to about 45 mg per tablet. Film-coated tablets were obtained with a release profile corresponding to the gastro-resistance indications of the European Pharmacopoeia.

The tablets of composition 5 have swollen and deformed, while all the others are stable.

### EXAMPLE 11

### Drying of the Compositions with CBD

It was operated as described in Example 3, but the step of mixing the active substances was carried out under vacuum, as described in Example 11. Samples taken during processing gave the analysis reported in the table.

TABLE 4
residual humidity in ademetionine and CBD compositions

| Sample | Phase | KF |
|---|---|---|
| Ademetionine powder | dosage | 2.7% |
| Composition of ademetionine + | mixing | 4.3% |
| CBD + fatty matter | | |
| Composition of ademetionine + | screening | 1.3% |
| CBD + fatty matter | | |

KF: residual humidity in % w/w according to Karl-Fisher.

The data show that, by carrying out the process under vacuum, a drying of the composition of ademetionine, cannabidiol and fatty matter can be obtained.

### EXAMPLE 12

### Further Compositions

Further compositions of ademetionine and CBD were prepared, by operating as described above but using the materials reported in the following table.

TABLE 5
Further compositions of ademetionine and CBD

| Test | Composition | Amount |
|---|---|---|
| I | Ryoto sugar ester P-1670 ® (E473) | 20 g |
| | Sunflower lecithin | 10 g |
| | CBD | |
| L | Ryoto sugar ester P-1570 ® (E473) | 20 g |
| | Sunflower lecithin | 10 g |
| | CBD | |
| M | Geleol NMB ® (E471) | 20 g |
| | Sunflower lecithin | 10 g |
| | CBD | |
| N | Ligamed SA-1-V ® | 20 g |
| | Sunflower lecithin | 10 g |
| | CBD | |
| O | Admul MG ® (E471) | 20 g |
| | Sunflower lecithin | 10 g |
| | CBD | |

Where: Ryoto sugar ester P-1670® and P-1570 are mixtures of mono- and tri-glycerides of fatty acids, Geleol NMB® is a glyceryl palmite stearate, Ligamed SA-1-V® is a mixture of stearic and palmitic acid. All excipients at room temperature are presented as waxes or solids with a low melting point (<70° C.). For the preparation it is operated as described in Example 1, by heating until the fatty matter melts before adding ademetionine.

All the preparations obtained (granulates) have proved to be low hygroscopic, flowable and easy to process.

The stability of ademetionine was verified in a similar way to that reported in experiment 2: in all preparations the residual ademetionine titre after 3 months is greater than 97% of the initial titre.

### EXAMPLE 13

### Compositions of Ademetionine and CBD with Polyethylene Glycols

Compositions of ademetionine, cannabidiol and fatty matter are prepared, by operating as described in Example 2 using the salt of ademetionine 51.5% (480 g) and pure CBD 98% (20.4 g). The ingredients reported in the following table were used as the fatty matter.

TABLE 6
Compositions of ademetionine, CBD and polyethylene glycols

| Test | Composition | Amount |
|---|---|---|
| P | Polyethylene glycol 4000 | 20 g |
| | Sunflower lecithin | 10 g |
| | CBD | |
| Q | Polyethylene glycol 6000 | 20 g |
| | Sunflower lecithin | 10 g |
| | CBD | |
| R | Stearin | 10 g |
| | Polyethylene glycol 4000 | 10 g |
| | Sunflower lecithin | 10 g |
| | CBD | |

All the preparations obtained were granulated and have proved to be low hygroscopic, flowable and easy to process.

The stability of ademetionine was verified in a similar way to that reported in experiment 2: in all preparations the residual ademetionine titre after 3 months is greater than 97% of the initial titre.

1-13. (canceled)

14. A pharmaceutical composition for oral use, comprising: ademetionine and cannabidiol and at least one pharmaceutically acceptable fatty matter.

15. The composition of claim 14, wherein said fatty matter has a melting point equal to or higher than +30° C.

16. The composition according to claim 15, wherein said fatty matter is chosen from a mono-, di-, tri-glyceride of a fatty acid, a polyether, or mixtures thereof.

17. The composition of claim 14, wherein ademetionine is present in an amount between 20% and 50% of the total weight of the composition, cannabidiol is present in an amount between 1% and 20% of the total weight of the composition, and the fatty matter is less than or equal to 35% of the total weight of the composition.

18. The composition according to claim 14, wherein the ratio of ademetionine to cannabidiol by weight is between 25:1 and 1:1.

19. The composition according to claim 14, further comprising a tetrahydrocannabinol content of less than 0.5% of the total weight of the composition.

20. The composition according to claim 14, further comprising at least one pharmaceutically acceptable emulsifier.

21. The composition according to claim 14, further comprising at least one pharmaceutically acceptable excipient comprising a polyglycol or an ester thereof with a fatty acid.

22. Pharmaceutical formulations in the form of tablets, coated tablets, capsules, stick-packs, containing the composition of claim 14.

23. A method for preparing a pharmaceutical composition, comprising the steps of:
a. dosing at least one fatty matter,
b. dosing cannabidiol,
c. dissolving cannabidiol in the fatty matter,
d. dosing ademetionine, and
e. mixing ademetionine with the mixture obtained in step c).

24. The method according to claim 22, wherein said ademetionine is mixed with a molten fatty matter.

25. The method according to claim 22, wherein said fatty matter is chosen from a fatty acid or an ester thereof, a mono-, di-, tri-glyceride of a fatty acid, a polyglycol, or mixtures thereof, having a melting point of less than +70° C.

26. Use of the compositions of claim 14 for oral administration of ademetionine and cannabidiol.
